(19) United States
(12) Patent Application Publication
(10) Pub. No.: US 2003/0139645 A1
(43) Pub. Date: Jul. 24, 2003

(54) Rotational freedom for a body organ

(76) Inventor: Thomas G. Adelman, West Baldwin, ME (US)

Correspondence Address: SHUMAKER & SIEFFERT, P. A., 8425 SEASONS PARKWAY, SUITE 105, ST. PAUL, MN, 55125, US

(21) Appl. No.: 10/057,754

(22) Filed: Jan. 23, 2002

(51) Int. Cl. A61F 2/00

(52) U.S. Cl. 600/37

(57) ABSTRACT

The invention provides techniques for securing a manipulating device that holds a moving organ, such as a beating heart. The manipulating device may be held securely and yet accommodate the natural rotational motion of the organ. In an exemplary application of the invention, an manipulating device holds the heart by the apex. The manipulating device is coupled to a support shaft, which is coupled to a key. The key is shaped so that it can engage a socket of a keyway in a securing structure. When the key engages the socket, the securing structure supports the key, which in turn supports the support shaft, the manipulating device and the heart. The rotational freedom of the heart is accommodated by, for example, allowing the support shaft to twist or by allowing the key to rotate in the socket.

### TECHNICAL FIELD

The invention relates to devices capable of providing adherence to organs of the body for purposes of medical diagnosis and treatment. More particularly, the invention relates to devices capable of adhering to, holding, moving, stabilizing or immobilizing an organ.

### BACKGROUND

In many areas of surgical practice, it may be desirable to manipulate an internal organ without causing damage to the organ. In some circumstances, the surgeon may wish to turn, lift or otherwise reorient the organ so that surgery may be performed upon it. In other circumstances, the surgeon may simply want to move the organ out of the way. In still other cases, the surgeon may wish to hold the organ, or a portion of it, immobile so that it will not move during the surgical procedure.

Unfortunately, many organs are slippery and are difficult to manipulate. Holding an organ with the hands may be undesirable because of the slipperiness of the organ. Moreover, the surgeon's hands ordinarily cannot hold the organ and perform the procedure at the same time. The hands of an assistant may be bulky, becoming an obstacle to the surgeon. Also, manual support of an organ over an extended period of time can be difficult due to fatigue. Holding an organ with an instrument may damage the organ, especially if the organ is unduly squeezed, pinched or stretched. Holding an organ improperly may also adversely affect the functioning of the organ.

The heart is an organ that may be more effectively treated if it can be manipulated. Many forms of heart manipulation may be useful, including moving the heart within the chest and holding it in place. Some forms of heart disease, such as blockages of coronary vessels, may best be treated through procedures performed during open-heart surgery. During open-heart surgery, the patient is typically placed in the supine position. The surgeon performs a median stemotomy, incising and opening the patient's chest. Thereafter, the surgeon may employ a rib-spreader to spread the rib cage apart, and may incise the pericardial sac to obtain access to the heart. For some forms of open-heart surgery, the patient is placed on cardiopulmonary bypass (CPB) and the patient's heart is arrested. Stopping the patient's heart is a frequently chosen procedure, as many coronary procedures are difficult to perform if the heart continues to beat. CPB entails trauma to the patient, with attendant side effects and risks. An alternative to CPB involves operating on the heart while the heart continues to beat.

Once the surgeon has access to the heart, it may be necessary to lift the heart from the chest or turn it to obtain access to a particular region of interest. Such manipulations are often difficult tasks. The heart is a slippery organ, and it is a challenging task to grip it with a gloved hand or an instrument without causing damage to the heart. Held improperly, the heart may suffer ischemia, hematoma or other trauma. The heart may also suffer a loss of hemodynamic function, and as a result may not pump blood properly or efficiently. Held insecurely, the heart may drop back into the chest, which may cause trauma to the heart and may interfere with the progress of the operation.

The problems associated with heart manipulation are greatly multiplied when the heart is beating. Beating causes translational motion of the heart in three dimensions. In addition, the ventricular contractions cause the heart to twist when beating. These motions of the heart make it difficult to lift the heart, move it and hold it in place.

In a coronary bypass operation, for example, the surgeon may need to manipulate the heart. The affected coronary artery may not be accessible without turning or lifting of the heart. Once the heart has been lifted or turned, the surgeon may need to secure the heart in a substantially fixed position.

### SUMMARY

In general, the invention provides techniques for securing a manipulating device that holds a moving organ, such as a beating heart. The manipulating device that holds the organ may be, for example, a device that holds the organ with vacuum pressure. The invention provides techniques for holding the manipulating device securely, thus limiting the motion of the organ to some degree, while simultaneously accommodating the natural motion of the organ. In particular, the invention accommodates rotational freedom of the organ.

In a representative application, the invention is directed to techniques for securing a manipulating device that holds the apex of a beating heart. As the heart beats, the heart bobs and twists. The twisting is problematic for at least two reasons. First, the twisting is important for the proper hemodynamic functioning of the heart, and therefore simply restraining the heart from all rotational motion has undesirable consequences upon hemodynamic functions. Second, the twisting compounds the difficulty of holding the heart with the manipulating device. The manipulating device may move and be difficult to control. Another potential difficulty is that the heart tissue may twist away from the manipulating device and may drop back into the chest or chafe against the manipulating device.

The invention addresses these concerns by accommodating some degree of rotational freedom of the heart. An organ support system supports the heart, yet allows the heart a degree of freedom to rotate. In one application of the invention, the heart is held by the apex with a vacuum-assisted manipulating device that includes a cup-like member and a skirt-like member. This manipulating device is coupled to a support shaft such as flexible vacuum tube. This application is merely exemplary. The invention is not limited to applications involving manipulation of the heart, nor is the invention limited to applications involving a vacuum-assisted manipulating device, nor is the invention limited to applications involving a manipulating device that is cup-shaped.

The support shaft extends through a securing structure and is coupled to a key. The key is shaped so that it can engage a keyway in the securing structure. The keyway may include a socket that is shaped to receive the key. The socket may be shaped so that the key may be received in more than one direction. When the key engages the keyway, the securing structure supports the key, which in turn supports the support shaft, the manipulating device and the heart. When the key engages the keyway, the keyway limits the rotational movement of the key relative to the securing structure. The organ support system does not necessarily restrain the motion of the heart. Some rotational motion may be permitted by, for example, the flexibility of the support shaft or a predetermined looseness in the engagement between the key and the keyway. Translational motion may be accommodated by a flexible support shaft or by a flexible coupling between the manipulating device and the support shaft.

In one embodiment, the invention is directed to a device comprising a manipulating device for contact with an organ, a support shaft coupled to the manipulating device and a key coupled to the support shaft. The support shaft may be flexible, and the key may be integrally formed with the support shaft. When the key engages a socket in a securing structure, the rotation of the key about an axis defined by the support shaft is restricted. The socket may include a ledge and a wall, the ledge restricting translational motion of the key and the wall restricting rotation of the key when the key is engaged in the socket.

In another embodiment, the invention is directed to a method comprising engaging a manipulating device with an organ. The manipulating device is coupled to a support shaft which in turn is coupled to a key. The method further comprises restricting the rotational movement of the key member relative to a securing structure. Rotational freedom may be provided to the organ by, for example, a flexible support shaft. The key may engage a socket in the securing structure snugly or with a predetermined looseness. Furthermore, the key may engage the socket in two or more directions, and the direction of engagement may be selected by the surgeon.

In a further embodiment, the invention presents a device comprising a supporting member and a collar coupled to the supporting member. The collar includes a socket shaped to engage a key that supports an organ. The socket may include a ledge that restricts the translational motion of the key and a wall that restricts rotation of the key when the key is engaged in the socket. The socket may engage the key snugly or loosely. The collar may further include an aperture that receives a support shaft coupled to the key member.

In an additional embodiment, the invention presents a method comprising engaging a manipulating device with an apex of a heart, the manipulating device coupled to a support shaft coupled to a key member. The method also includes engaging the key member with a socket in a securing structure. The heart may be lifted, and the load of the heart may be borne by the securing structure, key member, support shaft and manipulating device.

The invention can provide one or more advantages. The organ can be held securely in place, while simultaneously the organ can be allowed rotational freedom. In the context of heart surgery, the invention offers the surgeon access to a desired region of the heart while maintaining the hemodynamic functions of the heart.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a perspective view of a manipulation device and a support shaft in accordance with the invention, in conjunction with a beating heart, and with a key engaging a keyway.

FIG. 2 is a perspective view of the manipulation device and the support shaft shown in FIG. 1, with the key and keyway engaged.

FIG. 3 is a cross-sectional side view of the manipulation device and the support shaft shown in FIG. 1, with the key and keyway disengaged.

FIG. 4 is a cross-sectional side view of the manipulation device and the support shaft shown in FIG. 1, with the key and keyway engaged.

FIG. 5 is a cross-sectional side view of another manipulation device and a support shaft in accordance with the invention.

FIG. 6 is a plan view of an exemplary keyway socket in a collar with the cross-section of a corresponding key.

FIG. 7 is a plan view of another exemplary keyway socket that may correspond to the exemplary key shown in FIG. 6, with the cross-section of an alternate corresponding key.

FIG. 8 is a plan view of another exemplary keyway socket that may correspond to the exemplary key shown in FIG. 6.

FIG. 9 is a plan view of another exemplary keyway socket in a collar with the cross-section of a corresponding key.

FIG. 10 is a cross-sectional view of a coupling mechanism, illustrating an assembly technique.

FIG. 11 is a perspective view of a manipulating device, support shaft, key and collar illustrating an alternative assembly technique.

FIG. 12 is a plan view of a two-piece collar illustrating an alternative assembly technique.

FIG. 13 is a plan view of a slotted collar illustrating an alternative assembly technique.

### DETAILED DESCRIPTION

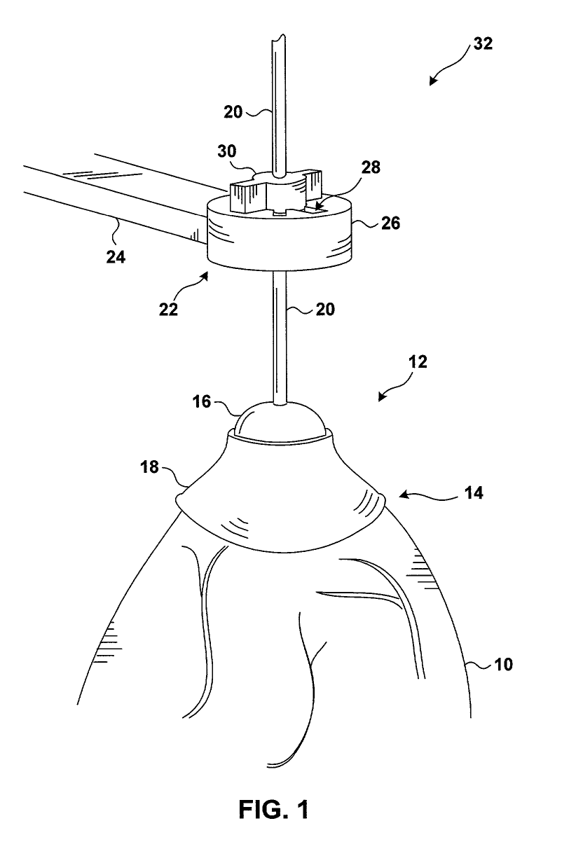

FIG. 1 is a perspective view of a heart 10, which is being held by a manipulating device 12. In the exemplary application shown in FIG. 1, a surgeon (not shown in FIG. 1) has obtained access to heart 10 and has placed manipulating device 12 over the apex 14 of heart 10. The surgeon has lifted apex 14 with manipulating device 12, giving the surgeon access to a desired region of heart 10. Although held by manipulating device 12, heart 10 has not been arrested and continues to beat. Beating causes heart 10 to move in three dimensions. In particular, heart 10 moves in translational fashion, by bobbing up and down and by moving from side to side. Heart 10 also expands and contracts as heart 10 fills with and expels blood. Heart 10 may twist as it expands and contracts.

Manipulating device 12 may engage heart 10 using any of a number of techniques. In FIG. 1, manipulating device 12 is an exemplary device that includes a cup-like member 16 and a skirt-like member 18 extending outward from cup-like member 16. Manipulating device 12 adheres to apex 14 with the aid of vacuum pressure supplied from a vacuum source (not shown in FIG. 1) via a vacuum tube 20, which may be formed integrally with cup-like member 16. Skirt-like member 18 deforms and substantially forms a seal against the surface of the tissue of heart 10. Skirt-like member 18 is formed of a compliant material that allows the seal to be maintained even as heart 10 beats. Adherence between heart 10 and manipulating device 12 may be promoted by other factors as well, such as a tacky surface of skirt-like member 18 placed in contact with heart 10.

Manipulating device 12 and vacuum tube 20 illustrate the practice of the invention. The invention is not limited to manipulating device 12, however. The invention may be practiced with a manipulating device that is not vacuum-assisted, or a manipulating device that is not cup-shaped, or a manipulating device that lacks a skirt-like member. The invention may be practiced with manipulating devices of any shape. For example, the invention may be practiced with a manipulating device that is irregularly shaped, including projections that extend radially outward from the center of the manipulating device and conform to the irregular shape of heart 10. In another context, the manipulating device may include a plurality of vacuum-assisted appliances, or the manipulating device may use no vacuum pressure at all.

In the exemplary application shown in FIG. 1, vacuum tube 20 serves as a support shaft for manipulating device 12 and as a supply of vacuum pressure. When manipulating device 12 is not vacuum-assisted, vacuum tube 20 may be replaced by a support shaft such as a plastic shaft. Alternatively, manipulating device 12 may be vacuum-assisted, but may be supported by a dedicated support shaft, with vacuum tube 20 providing little or no load-bearing support. The support shaft may be flexible.

The surgeon may move heart 10 by moving manipulating device 12 and/or vacuum tube 20. When the surgeon has obtained access to certain areas of heart 10, the surgeon may desire to maintain heart 10 in a substantially fixed position. In the exemplary application shown in FIG. 1, the surgeon suspends heart 10 by apex 14 and prepares to hold heart 10 in place with a securing structure 22.

Securing structure 22 includes a support arm 24 and a collar 26. Support arm 24 may be rigid or may be a adjustable arm that can be locked in a variety of positions. Support arm 24 may be affixed to a relatively immovable object, such as a rib spreader (not shown) or an operating table (not shown). Vacuum tube 20 passes through an opening, or keyway 28, in collar 26. Key 30 is coupled to vacuum tube 20. In one embodiment of the invention, key 30 is formed integrally with vacuum tube 20. Key 30 may also be affixed to vacuum tube 20 so as not to move relative to vacuum tube 20.

Key 30 is shaped so that key 30 engages keyway 28. In FIG. 1, key 30 is depicted as engaging keyway 28, but key 30 is not fully engaged with keyway 28. As will be shown in more detail below, keyway 28 includes a socket that is shaped to accommodate key 30. The socket does not penetrate through collar 26, so key 30 cannot pass through collar 26. An aperture permits passage of vacuum tube 20 but not key 30. As will be shown below, keyway 28 and key 30 may be shaped so that key 30 may engage keyway 28 in more than one way.

Manipulating device 12, a support shaft such as vacuum tube 20, key 30, support arm 24, and collar 26 with keyway 28 are components of an organ support system 32. Support system 32 holds heart 10, restraining the movement of heart 10. Support system 32 does not restrain all movement of heart 10, however, but permits some rotational freedom and some translational freedom as well.

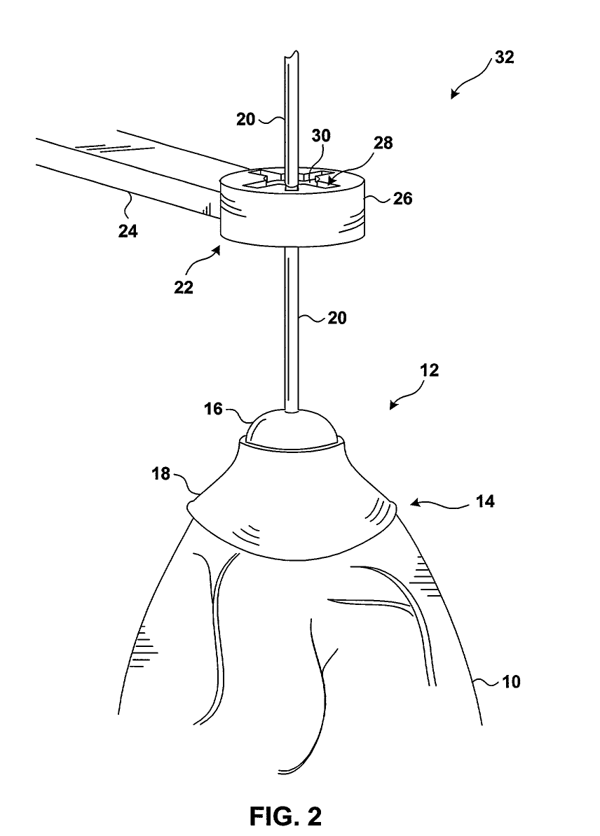

FIG. 2 is a perspective view similar to FIG. 1, except that key 30 is fully engaged with keyway 28, and support system 32 is bearing the load of heart 10. Collar 26 and support arm 24 support key 30, which rests in the socket of keyway 28. Key 30 supports vacuum tube 20, vacuum tube 20 supports manipulating device 12, and manipulating device 12 supports heart 10. The weight of heart 10 pulls on manipulating device 12 and vacuum tube 20, which is restrained from downward movement by the engagement of key 30 in keyway 28 of collar 26. In this way, support arm 24, collar 26, key 30, vacuum tube 20 and manipulating device 12 cooperate to bear the load of heart 10. Heart 10 continues to beat, and is held in tension by its own weight, which is borne by key 30 resting in the socket of keyway 28.

The engagement of key in keyway 28 prevents key 30 from rotating relative to collar 26. In other words, the rotation of key 30 about a longitudinal axis defined by vacuum tube 20 is restricted when key 30 engages keyway 28. Accordingly, vacuum tube 20 is constrained from rotating relative to collar 26. A support shaft such as vacuum tube 20 may be, but need not be, formed from flexible or semi-rigid materials that are strong in tension yet accommodate a degree of twisting and translational movement. As heart 10 beats, vacuum tube 20 may twist and bend to accommodate some rotational motion of heart 10. Heart 10 is thereby restrained and held in a substantially fixed position, yet continues to beat and is permitted sufficient rotational freedom of movement with each beat. As a result, the hemodynamic functions of heart 10 are preserved. In particular, the surgeon may maintain heart 10 in the desired position without stopping heart 10 and without causing a drop in aortic blood pressure.

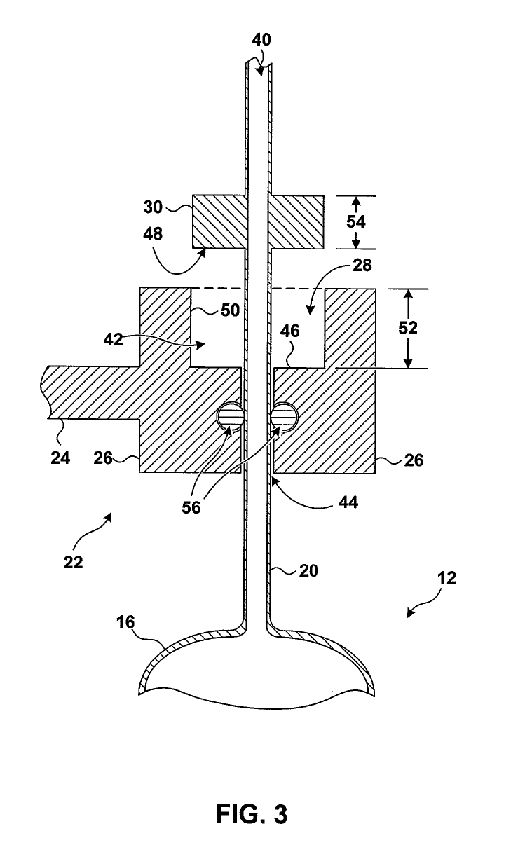

FIG. 3 is a cross-section of vacuum tube 20 extending through keyway 28 of collar 26. Vacuum tube 20 includes lumen 40. Vacuum pressure may be applied through lumen 40 to cause tissue (not shown in FIG. 3) to form a seal with manipulating device 12.

In FIG. 3, key 30 is shown as formed integrally with vacuum tube 20. Key 30 is not engaged with keyway 28 of collar 26. Keyway 28 includes a socket 42 and an aperture 44. Socket 42 includes a ledge 46, which supports the underside 48 of key 30. Socket 42 also includes a wall 50 that constrains the translational and rotational motion of key 30 when key 30 is engaged with keyway 28. Socket 50 has a depth 52 sufficient to prevent key 30 from disengaging from keyway 28 due to up-and down motion of the organ. In the embodiment depicted in FIG. 3, depth 52 of socket 42 is greater than the thickness 54 of key 30, but socket 42 need not be deeper than key 30 is thick.

Vacuum tube 20 is free to move up and down through aperture 44. Collar 26 includes rotational supports 56 to reduce friction between vacuum tube 20 and collar 26 when vacuum tube 20 moves in aperture 44. In particular, rotational supports 56 permit vacuum tube 20 to rotate and/or twist in aperture 44. In FIG. 3, rotational supports 56 are ball bearings.

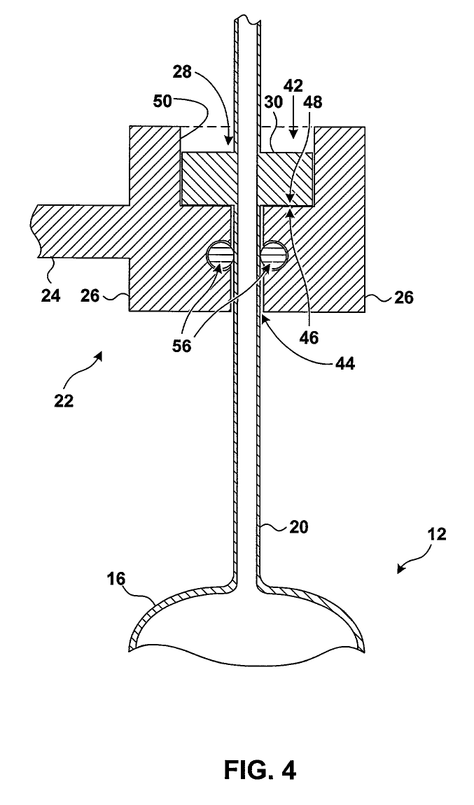

FIG. 4 is a cross-sectional view like FIG. 3, except FIG. 4 shows key 30 engaged with keyway 28. Manipulating device 12 may be engaged to an organ such as apex 14 of heart 10. The organ represents a load that pulls down on manipulating device 12. Support arm 24, collar 26, key 30, vacuum tube 20 and manipulating device 12 cooperate to bear the load of the organ. In particular, ledge 46 of socket 42 supports underside 48 of key 30. Key 30 in turn supports vacuum tube 20 and manipulating device 12. Wall 50 constrains the translational and rotational motion of key 30. Vacuum tube 20, however, may accommodate some rotational motion.

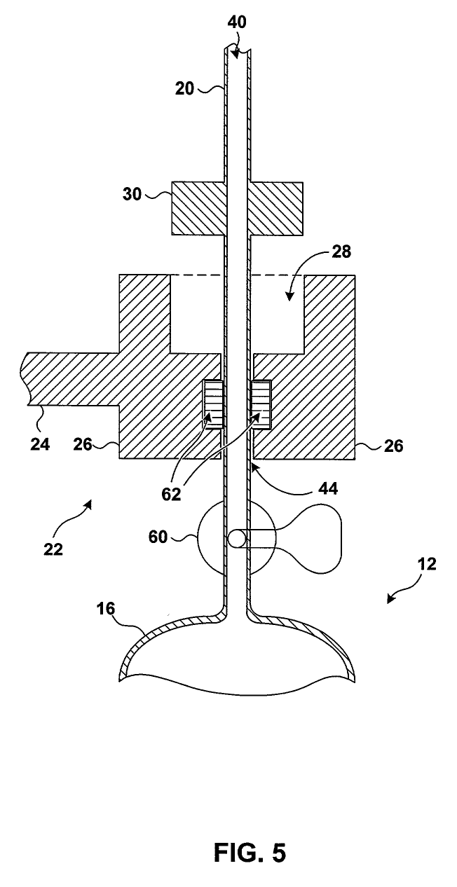

FIG. 5 shows an alternate embodiment of the invention. Unlike the embodiment shown in FIGS. 3 and 4, the embodiment shown in FIG. 5 includes a valve such as stopcock 60, to allow or prevent air from moving through vacuum tube 20. When vacuum pressure is applied via lumen 40, vacuum pressure may be maintained by shutting stopcock 60. Alternatively, the organ may be moved into engagement with manipulation device 12 by the surgeon, thereby expelling air through open stopcock 60 and through vacuum tube 20. Closing stopcock 60 prevents air from entering manipulation device 12 via vacuum tube 20, and may create a partial vacuum or negative pressure in manipulation device 12 without the need for an applied vacuum. Stopcock 60 may also be used to release vacuum pressure, to allow the organ to disengage from manipulation device 12.

Like the embodiment shown in FIGS. 3 and 4, the embodiment shown in FIG. 5 includes rotational supports 62. Rotational supports 62 may be bushings, and serve substantially the same purpose as the ball bearings 56 shown in FIG. 3.

The invention is not limited to use with vacuum-assisted manipulating device 12 as shown in FIGS. 1-5. Manipulating device 12 may include, for example, a frame or cradle that engages heart 10. When no vacuum is employed, vacuum tube 20 may be replaced by a support shaft that does not include a lumen 40. The support shaft may be, but need not be, flexible.

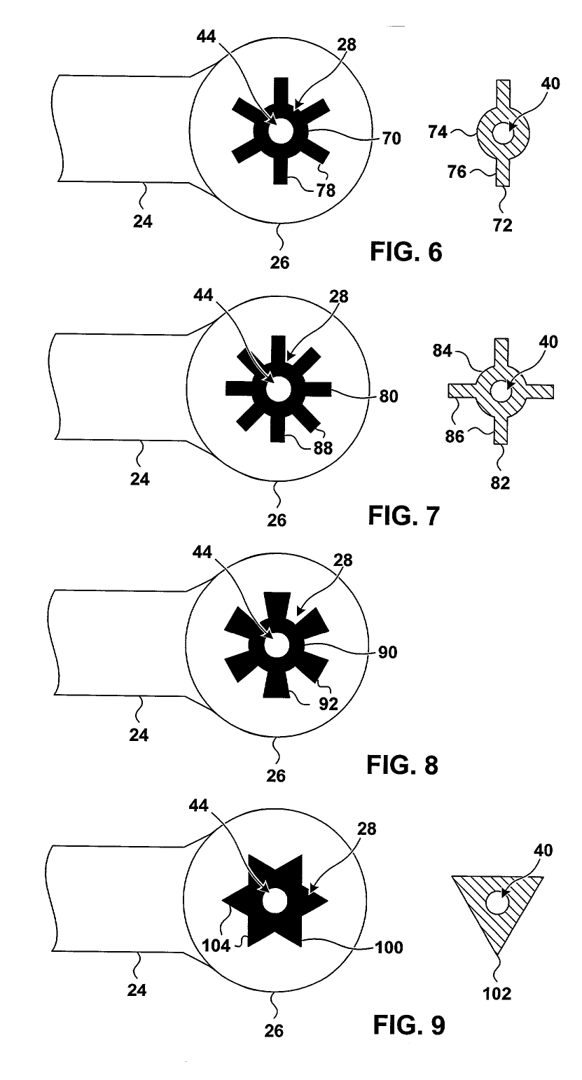

FIG. 6 is a plan view of collar 26 with keyway 28 having an exemplary socket 70. Exemplary key 72, shown in cross-section, fits socket 70. Key 72 does not represent the only possible key that may engage socket 70. Key 72 comprises a circular body 74 with two projections 76 extending radially away from body 74. Socket 70 includes recesses 78 that receive projections 76. Once key 72 engages keyway 28 (as illustrated in FIG. 1), projections 76 of key 72 mate with recesses 78 of socket 70, preventing rotation of key 72 relative to collar 26. Exemplary key 72 engages keyway 28 in any of six possible directions.

FIG. 7 is a plan view of collar 26 with an alternate keyway 28 having an exemplary socket 80. Socket 80 may engage an alternate exemplary key 82, shown in cross-section. Exemplary key 82 comprises a circular body 84 with four projections 86 extending radially away from body 84. Socket 80 includes recesses 88 that receive projections 86. Key 82 is not the only key that can engage socket 80, however. Exemplary key 72, shown in FIG. 6, also can engage socket 80.

Socket 80, like socket 70 shown in FIG. 6, may engage key 72 or key 82 in several different directions. Unlike socket 70, which includes six recesses 78, socket 80 includes eight recesses 82. Socket 80 may engage exemplary key 72 or exemplary key 82 in any of eight possible directions. Socket 70, by contrast, may engage exemplary key 72 in any of six possible directions. Socket 70 is not shaped to accommodate exemplary key 82 in any direction.

The number of possible directions of engagement of a key and a socket is for the convenience of the surgeon. The invention encompasses keys that engage sockets in any number of ways. In general, the more ways that a key may engage a keyway, the more freedom the surgeon has in positioning the key relative to collar 26. When a key engages a socket in only one way, for example, the surgeon must orient the key in a particular direction so that the key will engage the socket. This maneuver may result in an inconvenient arrangement and may also result in twisting of vacuum tube 20 or other support shaft, thereby unduly limiting the rotational freedom of heart 10 and compromising the hemodynamic functions of heart 10. Sockets 70 and 80 are exemplary sockets that offer the surgeon more options for positioning the key relative to collar 28 and avoid undesirable arrangements.

FIG. 8 is a plan view of collar 26 with an alternate keyway 28 having an exemplary socket 90. Socket 90 may accommodate keys of many shapes, such as exemplary key 72 shown in FIG. 6, and may engage the key in several different directions. Unlike sockets 70 and 80, socket 90 includes recesses 92 that are slightly flared. When a key such as exemplary key 72 engages socket 90, key 72 does not fit snugly in socket 90, but rather key 72 engages socket 90 with a predetermined looseness. In this manner, key 72 is permitted limited rotational freedom by the flared shape of recesses 92. Socket 90 may be used in an application in which vacuum tube 20 or other support shaft is rigid and does not accommodate twisting. In such an application, a degree of rotational freedom may be provided by the loose fit of key 72 in socket 90, in addition to or as an alternative to flexibility in the support shaft.

FIG. 9 is a is a plan view of collar 26 with keyway 28 having another exemplary socket 100. Exemplary key 102, shown in cross-section, fits socket 100, but exemplary key 102 is not the only possible key that may fit socket 100. Unlike keys 72 and 82 in FIGS. 6 and 7, key 102 does not include a circular body with projections. Rather, key 102 comprises a substantially equilateral triangle shape, and socket 100 includes recesses 104 that can receive key 102 in any of six possible directions. Once key 102 engages socket 100, the rotational freedom of key 102 relative to collar 26 is restricted.

The keys and sockets depicted in FIGS. 6, 7, 8 and 9 are illustrative of the kinds of keys and sockets that may be employed, but the invention is not limited to the particular configurations of keys and sockets shown. The variety of shapes of keys and sockets is unlimited, and the invention encompasses them all. Nor is the invention limited to any particular combination of key and socket. As has been demonstrated, a single key may correspond to several sockets, and a single socket may work with several keys.

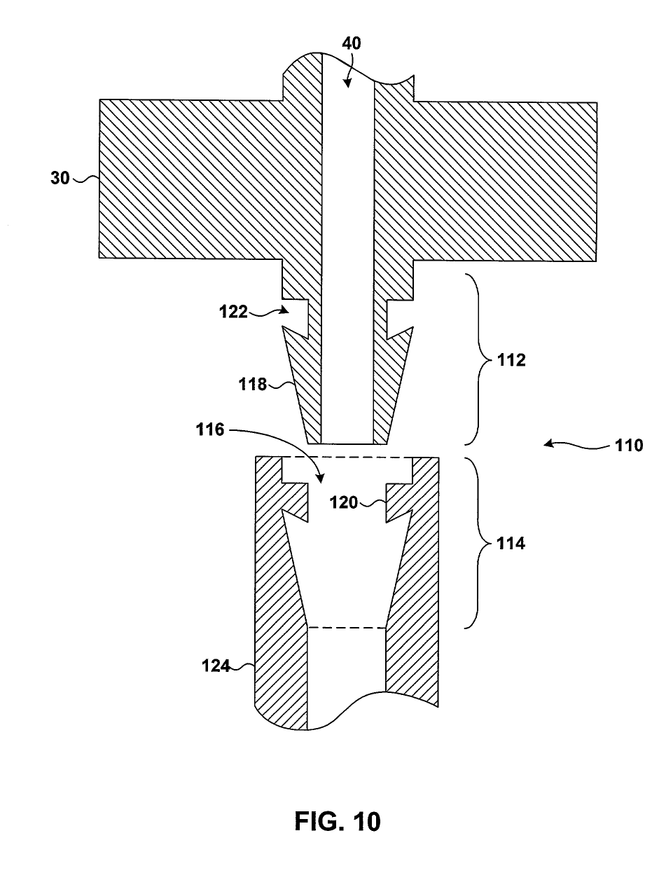

FIG. 10 is a cross-sectional view of a coupling mechanism 110 that may be included in an embodiment of the invention. In some embodiments of the invention, organ support system 32 requires assembly, and coupling mechanism 110 facilitates assembly. In the embodiment shown in FIG. 3, for example, vacuum tube 20 is enclosed in aperture 44, yet manipulating device 12 and key 30 are too large to fit through aperture 44. Coupling mechanism 110 provides one way to assemble organ support system 32.

Coupling mechanism 110 includes a male component 112 and a female component 114. Female component 114 includes an opening 116 that receives male component 112. Male component 112 includes a tapered end 118 for insertion into opening 116. When male component 112 is fully inserted into female component 114, male component 112 and female component 114 lock together.

In the embodiment of coupling mechanism 110 shown in FIG. 10, male component 112 and female component 114 lock together when a ridge 120 in female component 114 engages a notch 122 in male component 112. When ridge 120 engages notch 122, male component 112 and female component 114 are locked and are capable of bearing weight.

Male component 112, female component 114 or both may be formed from compliant material that may permit one or both components to deform so that male component 112 may be fully inserted into female component 114. In an exemplary construction, male component 112 is formed of a polymeric material of high durometer or of substantially rigid material, and female component 114 is formed of a polymeric material of a more flexible material. In this construction, female component 114 flares outward upon insertion of male component 112 and snap locks when ridge 120 engages notch 122.

Male component 112 and female component 114 may include a directional member (not shown in FIG. 10) that restricts how male component 112 may be locked to female component 114 and further prevents male component 112 from rotating relative to female component 114. Alternatively, male component 112 and female component 114 may lack a directional member and may be free to rotate relative to one another.

In the embodiment shown in FIG. 10, male component 112 of coupling mechanism 110 is proximal to key 30. Manipulating device 12 (not shown in FIG. 10) is coupled to a length of support shaft 124 that includes female component 114. Support shaft 124 may be threaded through aperture 44 of keyway 28 (not shown in FIG. 10), and male component 112 may be locked to female component 114. When key 30 is engaged in keyway 28, male component 112 and female component 114 are located in aperture 44. The narrowness of aperture 44 may restrain female component 114 from deforming when a load is applied to manipulating device 12, thereby securing the lock between male component 112 and female component 114. In this way, key 30, support shaft 124 and manipulating device 12 are assembled with collar 26. As shown in FIG. 10, coupling mechanism 110 may accommodate lumen 40 for supply of vacuum pressure, if needed.

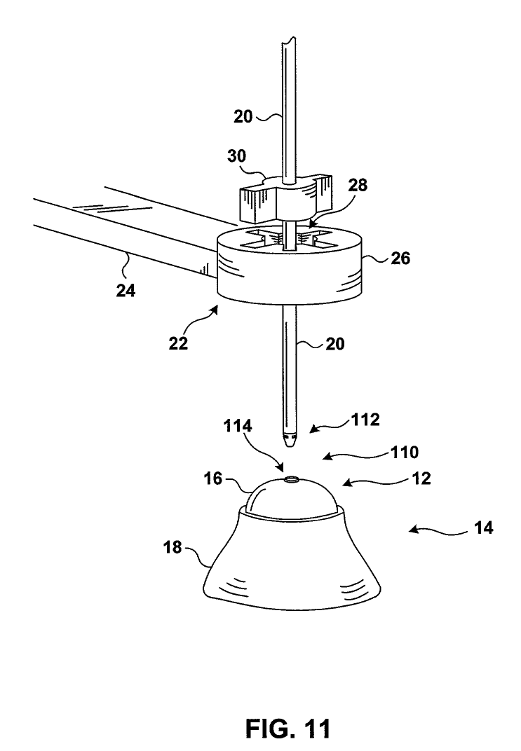

FIG. 11 depicts an alternative assembly technique. Vacuum tube 20 is threaded through keyway 28 and is coupled to manipulating device 12. Vacuum tube 20 includes male component 112, and manipulating device 12 includes female component 114. Male component 112 is inserted into female component 114. When male component 112 and female component 114 lock together, coupling mechanism 110 is capable of bearing weight.

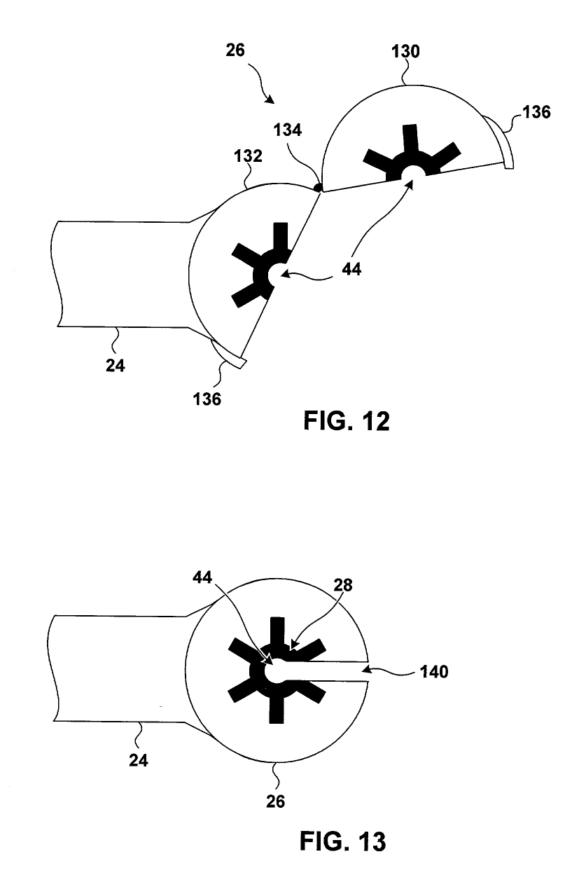

FIG. 12 illustrates another assembly technique. FIG. 12 shows collar 26 in two pieces 130 and 132, connected by hinge 134. Collar pieces 130 and 132 may be secured together with a latch 136. When secured together collar pieces 130 and 132 form a collar similar to the collar shown in FIG. 6. In this embodiment, vacuum tube 20 or other support shaft need not be threaded through aperture 44. Rather, key 30 and manipulating device 12 may be securely affixed to the support shaft, and collar 26 may be opened to receive the support shaft. Collar 26 may then be closed and secured with latch 136.

FIG. 13 illustrates an additional assembly technique. Collar 26 may include a slot 140 that allows the support shaft to be inserted into aperture 44. When key 30 engages keyway 28, the support shaft will be constrained from slipping through slot 140. In this embodiment, manipulating device 12, the support shaft and key 30 may be assembled prior to the surgical procedure, and may be slipped into collar 28.

The assembly techniques shown in FIGS. 10, 11, 12 and 13 are merely exemplary. Other assembly techniques may also be employed. For example, a support shaft may be threaded through aperture 44, and key 30 may be affixed to the support shaft. Coupling devices other than coupling mechanism 110 may be used, such as adhesives or heat bonding. The invention encompasses all of these variations.

The invention can provide one or more advantages. The heart can be manipulated and held in place so that the surgeon may have access to a desired region of the heart. Although the heart is held in place, the heart is granted rotational freedom so that the hemodynamic functions of the heart are preserved. As a result, the patient is less likely to suffer from circulatory problems during surgery.

Furthermore, the rotational freedom provided to the heart aids the manipulating device. Because the heart is allowed some freedom to twist, the heart is less likely to struggle against the manipulating device, thereby suffering ischemia, hematoma or other trauma. The heart is also more likely to be held securely and less likely to be dropped by the manipulating device.

Various embodiments of the invention have been described. These embodiments are illustrative of the practice of the invention. Various modifications may be made without departing from the scope of the claims. For example, the heart need not be held by the apex.

Moreover, the invention may be used to support the heart with the support shaft in a position other than vertical. In addition, the embodiments of the invention are not exclusive of other techniques for immobilizing a region of the heart, such as immobilizing an area around a vessel for bypass.

FIGS. 1 through 5 show manipulating device 12 and key 30 separated by an elongated support shaft or vacuum tube 20. These configurations are merely exemplary, and the invention is not limited to the configurations shown. The key may be closer to or farther from the manipulating device than is depicted in the figures. The invention encompasses support shafts of all lengths and all degrees of flexibility.

Furthermore, rotational freedom need not be provided exclusively by flexibility in the support shaft or by looseness in the engagement between the key and the keyway. Rotational freedom may be provided by both techniques simultaneously. Rotational freedom may also be provided by, for example, a swivel connection between the manipulating device and the support shaft, or a swivel connection between the support shaft and the key. The support shaft itself may include a flexible joint, swivel or other mechanism that provides rotational freedom.

Moreover, a vacuum-assisted manipulating device need not receive a supply of vacuum pressure via the support shaft, as shown in FIGS. 1 through 5. The support shaft may be, for example, a flexible tube with no lumen, and the vacuum supply may be provided to the manipulating device by a separate vacuum tube. The vacuum tube need not be load-bearing.

Although the embodiments are described in terms of heart surgery, the embodiments are not limited to use with the heart. Other organs may be held with a manipulating device and may be granted limited rotational freedom. The organs may be the subject of the surgical procedure, or they may held with a manipulating device so as to be out of the way of the surgical procedure.

The techniques for providing limited rotational freedom are not limited to the embodiments described above. Rotational freedom may be provided in other ways as well, such as by a swivel connection between the manipulating device and the support shaft, or by collar that can pivot relative to the support arm. These and other embodiments are within the scope of the following claims.

1. A device comprising:
a manipulating device having a surface to contact an organ;
a support shaft coupled the manipulating device; and
a key member coupled to the support shaft,
wherein the key member is shaped such that rotation of the support shaft about a longitudinal axis defined by the support shaft is restricted when the key member engages a socket in a securing structure.

2. The device of claim 1, wherein the support shaft is flexible.

3. The device of claim 1, wherein the support shaft comprises a vacuum tube.

4. The device of claim 1, further comprising:
the securing structure,
wherein the securing structure includes the socket, the socket shaped to engage the key member and including a ledge and a wall, the ledge restricting translational motion of the key member in at least one direction and the wall restricting rotation of the key member when the key member is engaged in the socket.

5. The device of claim 4, the supporting structure further including an aperture sized to permit passage of the support shaft.

6. The device of claim 4, the supporting structure further including:
an aperture sized to permit passage of the support shaft; and
a rotational support that bears against the support shaft.

7. The device of claim 4, the securing structure comprising a collar coupled to a support arm, the collar including the socket.

8. The device of claim 1, wherein the manipulating device is vacuum-assisted.

9. The device of claim 1, wherein the manipulating device is shaped to contact an apex of a heart.

10. The device of claim 1, wherein the key member comprises a central body surrounding the support shaft and a protrusion extending radially from the central body.

11. The device of claim 1, wherein the key member is integrally formed with the support shaft.

12. The device of claim 1, further comprising a coupling mechanism that couples the key member to the support shaft.

13. A method comprising:
engaging a manipulating device with an organ, the manipulating device coupled to a support shaft coupled to a key member; and
restricting the rotational movement of the key member relative to a securing structure.

14. The method of claim 13, wherein the support shaft is flexible, the method further comprising accommodating rotational freedom of the organ by allowing the support shaft to twist.

15. The method of claim 13, wherein restricting the rotational movement of the key member relative to the securing structure comprises engaging the key member in a socket in the supporting structure.

16. The method of claim 15, wherein the socket engages the key member with a predetermined looseness, the method further comprising accommodating limited rotational freedom of the organ by allowing the key member to rotate to a limited degree in the socket.

17. The method of claim 15, wherein the socket snugly engages the key member.

18. The method of claim 15, wherein the key member may engage the socket in at least two directions, and wherein engaging the key member in the socket comprises orienting the key member relative to the socket so that the key member engages the socket in one of the directions.

19. The method of claim 13, further comprising coupling the key member to the support shaft.

20. The method of claim 13, further comprising coupling the support shaft to the manipulating device.

21. The method of claim 13, wherein engaging the manipulating device with the organ comprises engaging the manipulating device with an apex of a heart.

22. The method of claim 21, further comprising holding the heart in tension with the manipulating device.

23. The method of claim 13, further comprising:
opening the securing structure to open an aperture;
receiving the support shaft in the aperture; and
closing the securing structure to close the aperture around the support shaft.

24. A device comprising:
a supporting member; and
a collar coupled to the supporting member, the collar comprising a socket, the socket shaped to engage a key member, the key member coupled to a support shaft and the support shaft coupled to a manipulating device that engages an organ, wherein the socket is shaped such that rotation of the key member about a longitudinal axis defined by the support shaft is restricted when the key member engages the socket.

25. The device of claim 24, wherein the socket includes a ledge and a wall, wherein the ledge restricts the translational motion of the key member and the wall restricts rotation of the key member when the key member is engaged in the socket.

26. The device of claim 24, wherein the socket is shaped to engage the key member snugly.

27. The device of claim 24, the collar further comprising an aperture that receives the support shaft.

28. The device of claim 27, further comprising a slot, the aperture configured to receive the support shaft via the slot.

29. The device of claim 27, wherein the collar comprises a first part and a second part, and wherein the aperture receives the support shaft when the first part separates from the second part.

30. The device of claim 24, wherein the socket comprises a plurality of projections.

31. The device of claim 24, wherein the socket is shaped to engage the key member in more than one direction.

32. A method comprising:
engaging a manipulating device with an apex of a heart, the manipulating device coupled to a support shaft and the support shaft coupled to a key member; and
engaging the key member with a socket in a securing structure.

33. The method of claim 32, further comprising lifting the apex of the heart.

34. The method of claim 32, further comprising bearing the load of the heart with the securing structure, key member, support shaft and manipulating device.

35. The method of claim 32, wherein the key member is configured to engage the socket in more than one direction.

36. The method of claim 32, further comprising accommodating rotational freedom of the heart by allowing the support shaft to twist.

37. The method of claim 32, further comprising accommodating rotational freedom of the heart by allowing the key member to rotate in the socket.
